(19) United States
(12) Patent Application Publication
(10) Pub. No.: US 2022/0061759 A1
(43) Pub. Date: Mar. 3, 2022

(54) NAVIGATION-BASED ECG RECORDING WITH A FEW ELECTRODES

(71) Applicants: Zepp, Inc., Cupertino, CA (US); Anhui Huami Health Technology Co., Ltd., Hefei (CN)

(72) Inventors: Artem Galeev, Vancouver (CA); Yan Vule, Port Moody (CA); Yoav Aminov, Tel Aviv (IL); Kongqiao Wang, Hefei (CN)

(21) Appl. No.: 17/004,556

(22) Filed: Aug. 27, 2020

(51) Int. Cl. A61B 5/00; A61B 5/0404

(52) U.S. Cl. CPC A61B 5/684; A61B 5/0404; A61B 5/0077; A61B 2562/0219; A61B 5/681

(57) ABSTRACT

A portable device for measuring an electrocardiogram (ECG) of a user. The portable device includes a first ECG sensor that is configured to touch a body of the user, and a processor. The processor is configured to receive a first ECG measurement at a first location on the body of the user, where the first ECG sensor is placed at the first location of the body; identify a first region of the heart of the user based on the first location; direct the user to move the portable device to a second location on the body of the user; receive a second ECG measurement at the second location on the body; and identify a second region of the heart of the user based on the second location.

### CROSS REFERENCES TO RELATED APPLICATION(S)

NONE.

### TECHNICAL FIELD

This disclosure relates generally to detecting physiological information of a user through a number of sensors of a wearable device, and more specifically to detecting blood pressure using, mainly, pressure sensors.

### BACKGROUND

Many portable devices have been developed in which some sensors can be used to detect variation in blood flow through arteries or blood volume in subcutaneous tissue and other sensors can be used to measure a heart's electrical activities. Applications include the monitoring of heart rate, glucose level, apnea, respiratory stress, and other physiological conditions.

High blood pressure is a major risk for heart disease. By some estimates, high blood pressure affects one in every three adults in the United States. By some other estimates, in developing and developed countries, respectively, 45% and 55% of high-blood-pressure sufferers are not aware of their condition.

Ischemia is another heart disease, which, if not monitored and treated, could lead to infraction (e.g., heart attacks). Ischemic heart disease can happen when one of the arteries that supplies the blood flow to the heart muscle is partially blocked. Ischemia of the tissue, if not treated, can further progress along to death or infarction of the tissue.

Early detection of ischemia and other conditions as well as the ability to monitor blood pressure using a portable (e.g., wearable) device are desirable.

### SUMMARY

Disclosed herein are implementations of a wearable device for measuring blood pressure.

A first aspect is a portable device for measuring an electrocardiogram (ECG) of a user. The portable device includes a first ECG sensor that is configured to touch a body of the user, and a processor. The processor is configured to receive a first ECG measurement at a first location on the body of the user, where the first ECG sensor is placed at the first location of the body; identify a first region of the heart of the user based on the first location; direct the user to move the portable device to a second location on the body of the user; receive a second ECG measurement at the second location on the body; and identify a second region of the heart of the user based on the second location.

A second aspect is a method for measuring an electrocardiogram (ECG) of a user. The method includes obtaining, using a portable device comprising a first ECG sensor, a first ECG measurement at a first location of a body of the user; identifying a movement of the portable device on the body of the user; identifying a second location of the body of the user based on the movement of the portable device; and obtaining, using the portable device, a second ECG measurement at the second location of the body.

A third aspect is a system for measuring an electrocardiogram (ECG) of a user. The system includes a portable device that includes a first ECG sensor, and an external device that is communication with the portable device. The portable device is configured to obtain a first ECG measurement at a first location of a body of the user; identify the first location based on sensor information received from the external device; prompt the user to move the portable device to a second location of the body of the user; and obtain a second ECG measurement at the second location of the body.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 depicts some aspects of an illustrative implementation of an apparatus according to implementations of this disclosure.

FIG. 2 depicts some aspects of an illustrative implementation of an apparatus according to implementations of this disclosure.

FIG. 3 depicts some aspects of an illustrative implementation of an apparatus according to implementations of this disclosure.

FIGS. 4A-4B depict some aspects of a user's anatomy according to implementations of this disclosure.

FIGS. 5A-5C depict some aspects of an illustrative implementation of an apparatus according to implementations of this disclosure.

FIG. 6A-6C depict some aspects of an illustrative implementation of an apparatus according to implementations of this disclosure.

FIG. 7 depicts some aspects of an illustrative implementation of an apparatus according to implementations of this disclosure.

FIG. 8 depicts an illustrative implementation of a computing system according to implementations of this disclosure.

FIG. 9A illustrates placement of precordial electrodes.

FIG. 9B illustrates regions of a traditional ECG associated with each of the precordial electrodes.

FIG. 10 illustrates examples of ECGs of normal and ischemic myocardium.

FIG. 11 is a flowchart of a technique for measuring an ECG of a user according to an implementation of this disclosure.

FIG. 12 illustrates an example of a user placing a wrist-worn portable device at the V1 location of FIG. 9A according to implementations of this disclosure.

FIG. 13 is a flowchart of another technique for measuring an ECG of a user according to an implementation of this disclosure.

### DETAILED DESCRIPTION

Disclosed herein are implementations of an apparatus for sensing, measuring, analyzing, and/or displaying physiological information. In one aspect, the apparatus may be a wearable device comprising an upper module and/or a lower module. The wearable device may be worn on a user's body such that one or more sensors of the upper and lower modules contact a targeted area of tissue. In one implementation, the wearable device is a watch, band, or strap that can be worn on the wrist of a user such that the upper and lower modules are each in contact with a side of the wrist.

In an embodiment, the wearable device can be a lower module that can be attached to another device. For example, the wearable device can be a lower module that is a clip and/or an add-on to a watch or another wearable device. For example, the lower module may be attachable to the bottom of a watch such that the lower module is in contact with the skin of the wearer.

Each of the upper and lower modules may comprise one or more sensors, including but not limited to optical/PPG sensors, Electrocardiogram (ECG) sensors/electrodes, bio impedance sensors, galvanic skin response sensors, tonometry/contact sensors, accelerometers, pressure sensors, acoustic sensors, electro-mechanical movement sensors, and/or electromagnetic sensors. In one implementation, one or more optical/PPG sensors may comprise one or more light sources for emitting light proximate a targeted area of tissue and one or more optical detectors for detecting either reflected light (where an optical detector is located on the same side of the targeted area as the light source(s), i.e., within the same module) or transmitted light (where an optical detector is located opposite the light source(s), i.e., within an opposing module).

In a further aspect, the strap or band of the wearable device may be configured so as to facilitate proper placement of one or more sensors of the upper and/or lower modules while still affording the user a degree of comfort in wearing the device. In one implementation, rather than a strap that lies in a plane perpendicular to the longitudinal axis of the user's wrist or arm (as is the case with traditional wrist watches and fitness bands), the band may be configured to traverse the user's wrist or arm at an angle that brings one or more components of the upper or lower modules into contact with a specific targeted area of the user while allowing another portion of the band to rest at a position on the user's wrist or arm that the user finds comfortable.

In another aspect, the precise location of the upper and/or lower modules can be customized such that one or more sensors of either module can be placed in an ideal location of a user, despite the physiological differences between body types from user to user.

The aforementioned features result in more comfortable wearable device while also increasing reliability and accuracy of the device sensing, measuring, analyzing, and displaying of physiological information.

In one implementation, the physiological information sensed, measured, analyzed, or displayed can include but is not limited to heart rate information, ECG waveforms, calorie expenditure, step count, speed, blood pressure, oxygen levels, pulse signal features, cardiac output, stroke volume, and respiration rate. In further implementations, the physiological information may be any information associated with a physiological parameter derived from information received by one or more sensors of the wearable device. Regardless, the physiological information may be used in the context of, for example, health and wellness monitoring, athletic training, physical rehabilitation, and patient monitoring. Of course, these examples are only illustrative of the possibilities and the device described herein may be used in any suitable context.

As illuded to above, any tissue organ in the body can experience ischemic changes and then become infarcted. An electrocardiogram, which is a graph of the electrical activity of the heart, can be used to detect cardiac abnormalities. Conventionally, a 12-lead ECG that uses 10 electrodes placed on a patient's body and connected via wires to a device that measures electrical activity is used. Additionally, with such a device, the electrodes are placed by a professional who is trained in the proper and optimal placement of the electrodes on the body. Thus, such a device is impractical for ambulatory use by the average person (e.g. user) who is interested in monitoring for abnormal heart conditions, such as ischemia.

Implementations according to this disclosure relate to a portable device, which can be a wearable device (such as a wrist-worn portable device) or a hand-portable device, that can be used by a user to measure electrical activity of the heartbeat of the user. The portable device includes an ECG sensor (e.g., an electrode) that the user can place on his/her chest or other body locations. The portable device (using a processor therein) can detect (e.g., determine, calculate, etc.) the location of the sensor on the body to determine a lead (e.g., an angle of measurement). In another example, the location of the sensor can be determined using sensors of a device that is external to the portable device. In an example, digital image processing can be used. For example, an external camera that can be used to image the chest of the user as the portable device is placed on the chest. The location of the portable device can be determined using an image of the camera.

In another implementation, the portable device can be a wearable device that includes a strap and that can be worn on a wrist of a first arm of the user. A second sensor can be included in the tail of the strap. While taking measurements, the user can touch the second sensor or hold the second sensor (such as between the thumb and index fingers of the other hand of the user). As such, the measurement can be more accurate when more electrodes (e.g., ECG sensors) are used.

These and other example configurations of the portable device are further described below.

While the systems and devices described herein may be depicted as wrist worn devices, one skilled in the art will appreciate that the systems and methods described below can be implemented in other contexts, including the sensing, measuring, analyzing, and display of physiological data gathered from a device worn at any suitable portion of a user's body, including but not limited to, other portions of the arm, other extremities, the head, and/or the chest.

Reference will now be made in detail to certain illustrative implementations, examples of which are illustrated in the accompanying drawings. Wherever possible, the same reference numbers will be used throughout the drawings to refer to the same or like items.

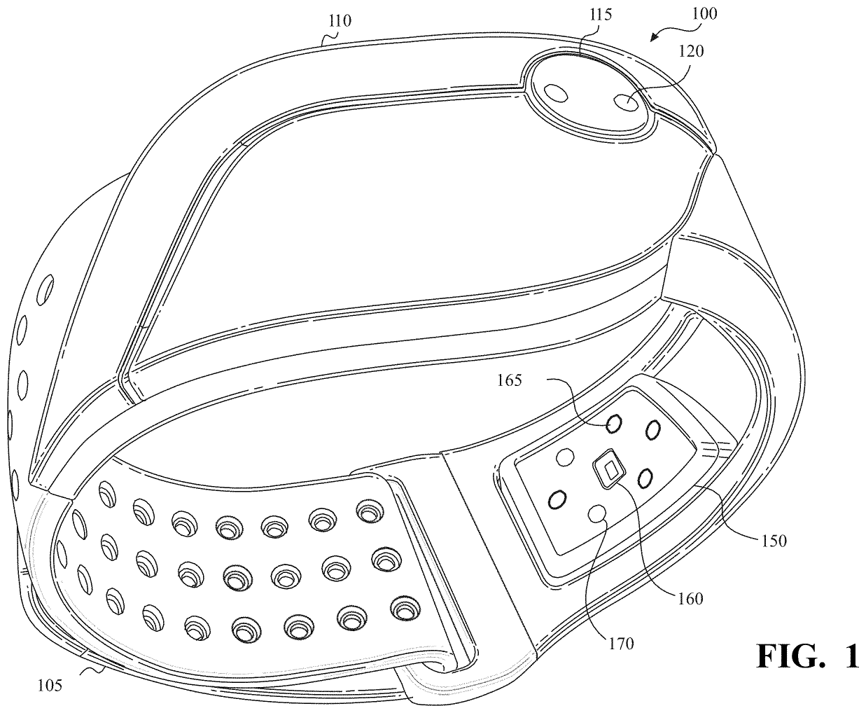

FIG. 1 depicts an illustrative implementation of an apparatus 100 according to implementations of this disclosure. In one aspect, apparatus 100 may be a physiological monitor worn by a user to sense, collect, monitor, analyze, and/or display information pertaining to one or more physiological parameters. In the depicted implementation, apparatus 100 may comprise a band, strap, or wrist watch. In further implementations, apparatus 100 may be any wearable monitor device configured for positioning at a user's wrist, arm, another extremity of the user, or some other area of the user's body.

In another aspect, apparatus 100 may comprise at least one of an upper module 110 or a lower module 150, each comprising one or more components and/or sensors for detecting, collecting, processing, and displaying one or more physiological parameters of a user and/or other information that may or may not be related to health, wellness, exercise, or physical training sessions.

In addition to upper module 110 and lower module 150, apparatus 100 may comprise a strap or band 105 extending from opposite edges of each module for securing apparatus 100 to the user. In one implementation, band(s) 105 may comprise an elastomeric material. In alternative implementations, band(s) 105 may comprise some other suitable material, including but not limited to, a fabric or metal material.

Upper module 110 or lower module 150 may also comprise a display unit (not shown) for communicating information to the user. The display unit may be an LED indicator comprising a plurality of LEDs, each a different color. The LED indicator can be configured to illuminate in different colors depending on the information being conveyed. For example, where apparatus 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees apparatus 100 through his or her peripheral vision.

In addition, or alternatively, the display unit may comprise a display screen for displaying images or characters to the user. The display unit may further comprise one or more hard or soft buttons or switches configured to accept input by the user.

Apparatus 100 may further comprise one or more communication modules. In some examples, each of upper module 110 and lower module 150 comprise a communication module such that information received at either module can be shared with the other module.

One or more communication modules can also be configured to communicate with other devices such as the user's cell phone, tablet, or computer. Communications between the upper and lower modules can be transmitted from one module to the other wirelessly (e.g., via Bluetooth, RF signal, Wi-Fi, etc.) or through one or more electrical connections embedded in band 105. In a further implementation, any analog information collected or analyzed by either module can be translated to digital information for reducing the size of information transfers between modules. Similarly, communications between either module and another user device can be transmitted wirelessly or through a wired connection, and translated from analog to digital information to reduce the size of data transmissions.

As shown in FIG. 1, lower module 150 can comprise an array of sensor array 155 including but not limited to one or more optical detectors 160, one or more light sources 165, and one or more contact pressure/tonometry sensors 170. These sensors are only illustrative of the possibilities, however, and lower module may comprise additional or alternative sensors such as one or more acoustic sensors, electromagnetic sensors, ECG electrodes, bio impedance sensors, galvanic skin response sensors, and/or accelerometers. Though not depicted in the view shown in FIG. 1, upper module 110 may also comprise one or more such sensors and components on its inside surface, i.e. the surface in contact with the user's tissue or targeted area.

In some implementations, the location of sensor array 155 or the location of one or more sensor components of sensor array 155 with respect to the user's tissue may be customized to account for differences in body type across a group of users. For example, band 105 may comprise an aperture or channel 175 within which lower module 150 is movably retained. In one implementation, lower module 150 and channel 175 can be configured to allow lower module 150 to slide along the length of channel 175 using, for example, a ridge and groove interface between the two components. In this manner, and as described in more detail below, where the user desires to place one more components of sensor array 155 at a particular location on his or her wrist, lower module 150 can be slid into the desired location along band 105. Though not depicted in FIG. 1, band 105 and upper module 110 can be similarly configured to allow for flexible or customized placement of one or more sensor components of upper module 110 with respect to the user's wrist or targeted tissue area.

In addition to the sensors and components proximate or in contact with the user's tissue, upper module 110 and/or lower module 150 may comprise additional sensors or components on their respective outer surfaces, i.e. the surfaces facing outward or away from the user's tissue. In the implementation depicted in FIG. 1, upper module 110 comprises one such outward facing sensor array 115. In one implementation, sensor array 115 may comprise one or more ECG electrodes 120. Similar to the sensor arrays of the upper and lower modules proximate or in contact with the user's tissue, outward facing sensor array 115 may further comprise one or more contact pressure/tonometry sensors, photo detectors, light sources, acoustic sensors, electromagnetic sensors, bio impedance sensors, galvanic skin response sensors, and/or accelerometers.

The outward facing sensors of sensor array 115 can be configured for activation when touched by the user (with his or her other hand) and used to collect additional information. For example, where lower module 150 comprises one or more optical detectors 160 and light sources 165 for collecting PPG and heart rate information of the user, outward facing sensor array 115 of upper module 110 may comprise ECG electrodes 120 that can be activated when the user places a fingertip in contact with the electrodes. While the optical detectors 160 and light sources 165 of lower module 150 can be used to continuously monitor blood flow of the user, outward facing sensor array 115 of upper module 110 can be used periodically or intermittently to collect potentially more accurate blood flow information which can be used to supplement or calibrate the measurements collected and analyzed by an inward facing sensor array, the sensor array 155, of lower module 150.

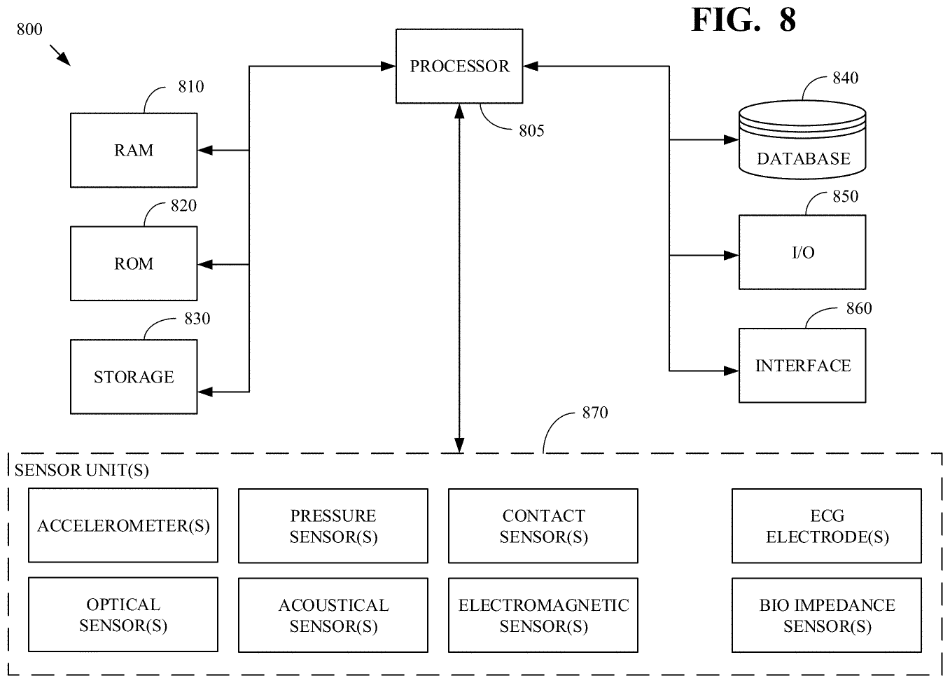

In addition to the sensor components described above with respect to each module, each module may further comprise other components for receiving, storing, analyzing, and/or transmitting physiological information. Some of those components are described below with respect to FIG. 8.

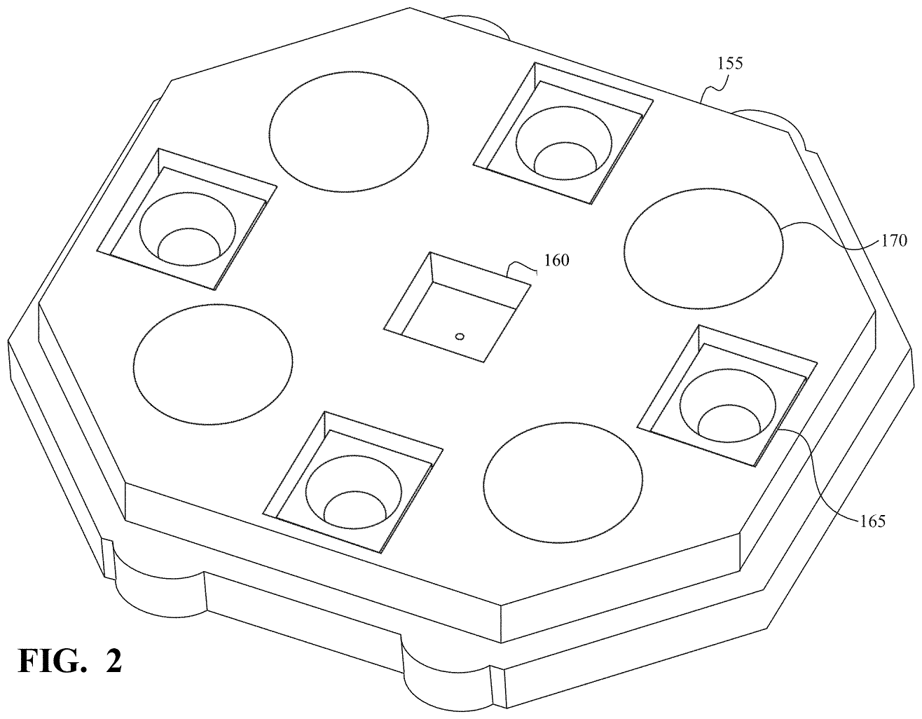

FIG. 2 depicts one implementation of inward facing sensor array, the sensor array 155, of lower module 150 according to implementations of this disclosure. As shown, sensor array 155 can comprise sensors including but not limited to one or more optical detectors 160, one or more light sources 165, and one or more contact pressure/tonometry sensors 170. These sensors are only illustrative of the possibilities, however, and sensor array 155 may comprise additional or alternative sensors such as one or more acoustic sensors, electromagnetic sensors, ECG electrodes, bio impedance sensors, galvanic skin response sensors, and/or accelerometers. Upper module 110 may comprise a similar inward facing sensor array (not depicted in FIG. 1) configured to position sensors proximate or in contact with the outside portion of a user's wrist or arm. In some implementations, sensor components of the upper module 110 and the lower module 150 can be used in combination to collect and analyze physiological information. For example, and as described in more detail below, one or more light sources of lower module 150 can be used to transmit light through a targeted area of the user's tissue (e.g., a portion of the user's wrist) and the transmitted light can be detected by one or more photodetectors of an inward facing sensor array of upper module 110. In such an implementation, opposing modules 110 and 150 can be used to detect and analyze either reflected or transmitted light.

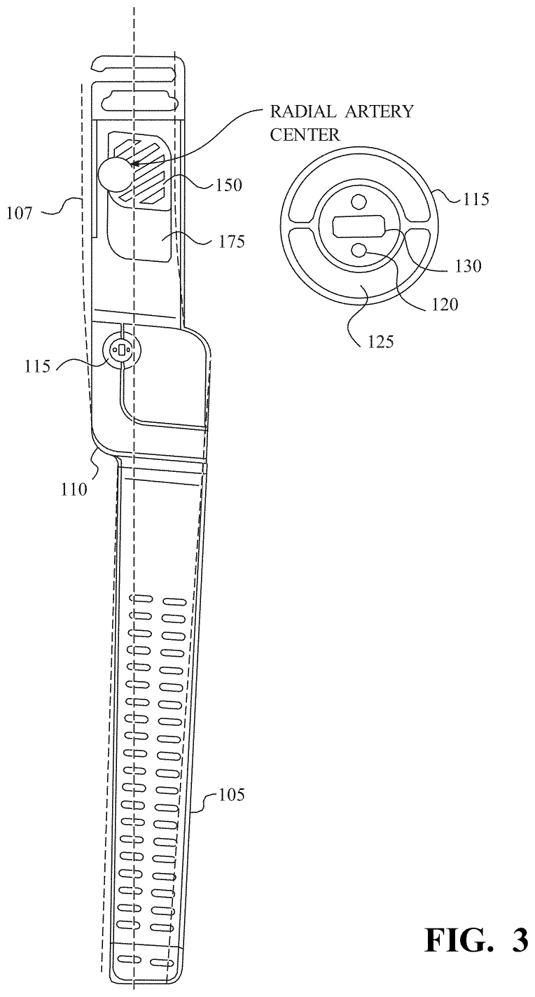

FIG. 3 depicts another view of apparatus 100 comprising band 105, upper module 110, and lower module 150 according to implementations of this disclosure. As described above, lower module 150 can be placed within channel 175 of band 105 such that lower module 150 can slide along the longitudinal axis of band 105. The movability of lower module 150 (or upper module 110 in alternative implementations) with respect to band 105 allows a user to customize the location of the inward facing sensors of lower module 150 with respect to a targeted tissue area to ensure reliable and accurate detection of physiological parameters. For example, a user can ensure that the inward facing sensors of lower module 150 are place in a location proximate the center of the user's radial artery.

In another aspect, band 105 may not extend around the user's wrist such that it traverses a circumferential path lying in a plane perpendicular to the longitudinal axis of the user's wrist or arm. Rather, the longitudinal axis of band 105 extends at an angle such that portions of inward facing sensor arrays of upper or lower modules 110, 150 can be placed at suitable locations proximate a desired targeted area of tissue while a portion of band 105 is in contact with portions of the user's wrist or arm that the user finds comfortable (i.e., above or below the wrist joint). In some implementations, where a circumferential path around a user's wrist resides in a plane perpendicular to the longitudinal extension of the user's arm or wrist, band 105 may be set at an angle 107 with respect to the perpendicular plane. In some implementations, angle 107 may be between 5° and 15° with respect to the perpendicular plane. In other implementations, angle 107 may be less than 5° or more than 15°. Of primary importance is the placement of one or more components of the sensor arrays of upper and lower modules 110, 150 proximate or in contact with a desired targeted area of tissue while allowing a portion of band 105 to be worn at a comfortable location off the user's wrist joint. Additional details regarding proper or desirable placement of one or more sensors with respect to targeted tissue areas of a user are described below with respect to other figures.

FIG. 3 also shows a closer view of outward facing sensor array 115. In the implementation depicted, sensor array 115 may comprise one or more ECG electrodes 120 for establishing an electrical connection with a user's fingertip and collected ECG data. Sensor array 115 may further comprise one or more contact pressure/tonometry sensors 125 for detecting the presence of the user's fingertip, which can trigger activation of the ECG electrodes 120. Sensor array 115 may also comprise additional or alternative components 130 such as one or more optical detectors, light sources, acoustic sensors, electromagnetic sensors, bio impedance sensors, galvanic skin response sensors, and/or accelerometers.

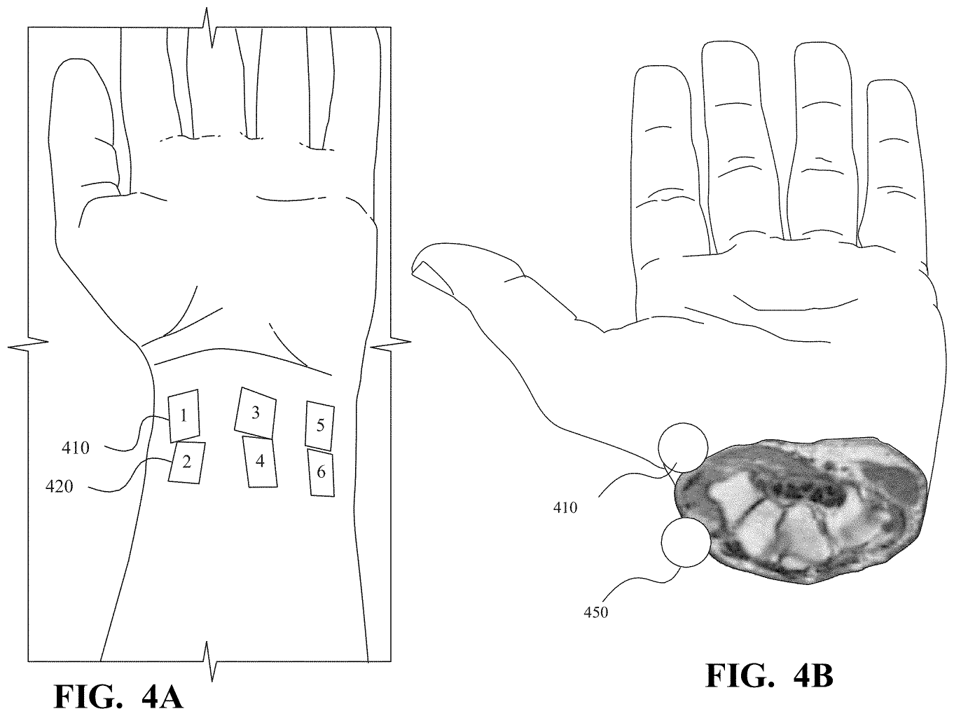

FIG. 4A depicts some points of interest on a human wrist. The best point on the wrist for detecting blood flow, for example in calculating heart rate, blood pressure, respiratory rate, etc., is at a location coinciding with the wrist joint, approximately at location 1 shown as item 410. This location is proximate the radial artery and is referred to as the CUN location.

Thus, the ideal location for a user to wear a wrist worn device is along the line across a line comprising locations 1-3-5. However, for comfort, most users prefer to wear straps or bands off the wrist joint, for example, across locations 2-4-6 shown as item 420. The result is that in most cases, users wear their monitors and corresponding sensors at a location on their wrist or arm that is not ideal and likely to introduce errors in the detection of physiological parameters.

The angle 107 of the band described with respect to FIG. 3 cures this deficiency in that it allows one or more sensor components of lower module 150 to be located above the CUN location while allowing a portion of the remaining band and/or upper module 110 to be positioned at a more comfortable location on the user's wrist or arm, such as line 2-4-6 (item 420).

Further ensuring that one or more sensors of lower module 150 can be placed at a desirable location above the CUN location, and as described in more detail above with respect to FIGS. 1 and 3, lower module 150 can slide along band 105. This allows the user to make further adjustments to the location of one or more sensors, not just along the longitudinal extension of the user's arm when apparatus 100 is in use, but also along the circumferential extension of band 105 while apparatus 100 is in use. Thus, the combination of band 105 extending around the user's wrist at an angle 107 together with the ability to slide the lower module 150 along band 105, ensure the sensors of lower module 150 can be placed at an ideal location with respect to each user (even users of different body types and physical attributes) and that the physiological parameters detected and analyzed by apparatus 100 are collected as accurately as possible.

Not only is apparatus 100 configured so as to ensure proper placement of one or more sensors and comfortability of band 105, but it also may contain additional sensors, such as a pressure sensor, at locations of apparatus 100 other than upper and lower modules 110, 150.

For example, apparatus 100 may comprise a pressure sensor located somewhere else along band 105 or at a latch that secures opposing ends of band 105 around a user's wrist for detecting pressure. Such a sensor can be used to ensure that the user is wearing the apparatus 100 tightly enough to ensure one more other sensors are in sufficient contact with a targeted area of the user's tissue to collect accurate physiological information. In alternative implementations, one or more pressure sensors of the upper and/or lower modules 110, 150 can be used to make the same determination. In either case, apparatus 100 may also be configured to alert the user (for example, via the display unit of upper module 110) if apparatus 100 is being worn too loosely or too tightly to ensure accurate measurements.

FIG. 4B depicts one example of desirable locations for one or more sensors to be placed with respect to a user's wrist or other targeted area according to implementations of this disclosure. In one implementation, one or more sensors of lower module 150 can be placed adjacent or proximate the item 410 (i.e., the CUN location) and one or more sensors of upper module 110 can be placed opposite the item 410 at point 450 of the user's wrist or targeted area. Such a configuration provides the aforementioned benefits associated with proper placement of sensors over the CUN location, but also allows for apparatus 100 to detect, collect, and analyze blood flow through the radial artery using either reflective or transmissive systems.

Wrist worn PPG sensors currently use a reflective system whereby a sensor array comprises one or more light sources and one or more optical detectors, located near one another and on the same side of a user's targeted area. The one or more light sources of the sensor array illuminate a portion of the user's tissue and light is reflected back to the optical detector(s) of the sensor array. The reflected light detected by the optical detector can be analyzed to estimate physiological parameters such as blood flow and pulse rate.

However, reflective systems may not be as accurate as transmissive systems that place one or more light sources on one side of a user's body and optical detectors on an opposing side of the user's body. One example of a transmissive system are fingertip monitors used in a clinical setting. The monitors are clipped to a patient's fingertip, one side comprising a light source for illuminating the top or bottom of the patient's fingertip, the other side comprising an optical detector for detecting the light transmitted through the fingertip.

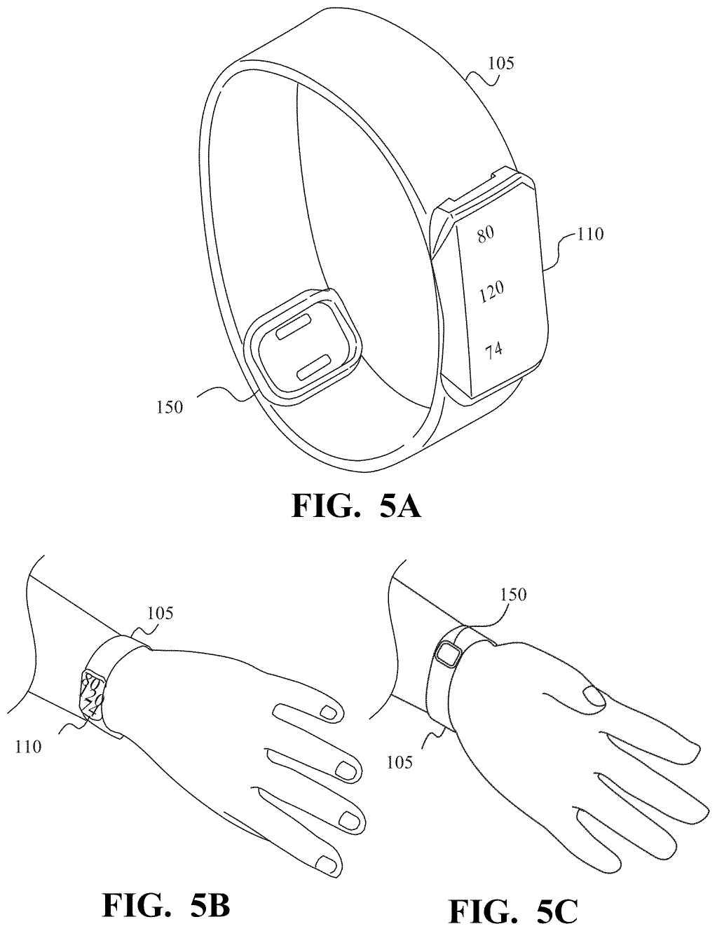

It has been thought that transmissive systems are not practical for wrist worn health monitors (or monitors worn at other locations on a user's arm or body) because the wrist is too thick for light that enters one side of a targeted area to be transmitted all the way through to the other side. However, apparatus 100 solves this problem by taking advantage of the natural location of the CUN location (the location of the radial artery at the wrist) at the inside of the wrist just under the thumb. As shown in FIGS. 5A, 5B, and 5C, apparatus 100 can be configured to place the lower module 150 comprising a light source (and/or optical detector) at the location of the CUN artery on the underside of the wrist and place the upper module 110 comprising an opposing optical detector (or light source) at a location opposite the sensors of the lower module at the periphery of the outside of the wrist just below the thumb. In this manner, the path of light transmitted through the wrist between the sensors of the lower and upper modules travels a shorter distance (shown in FIG. 4B) than if the sensors were located closer to the center of the inside and outside of a user's wrist. As a result, light illuminated from either the upper or lower module can be detected at the opposing module in a manner previously only available in clinical settings and limited to locations on the body such as the fingertip.

As described above, apparatus 100 may comprise a number of components and sensors for detecting physiological information and extracting data from it, such as blood flow, heart rate, respiratory rate, blood pressure, steps, calorie expenditure, and sleep.

Data collected from at least one or more of ECG electrodes/sensors, bio impedance sensors, galvanic skin response sensors, tonometry/contact sensors, accelerometers, pressure sensors, acoustic sensors, and electromagnetic sensors can be used for determining physiological information.

One method for determining the heart rate, respiratory rate, blood pressure, oxygen levels, and other parameters of a user involves collecting a signal indicative of blood flow pulses from a targeted area of the user's tissue. As described above, this information can be collected using, for example, a light source and a photo detector. Some implementations may use multiple light sources and they may be of varying colors (e.g., green, blue, red, etc.). For example, one light source may be an IR light source and another might be an LED light (such as a red LED). Using both an IR light source and a colored LED light (such as red) can improve accuracy as red light is visible and most effective for use on the surface of the skin while IR light is invisible yet effective for penetration into the skin. Such implementations may comprise multiple photo detectors, one or more configured to detect colored LED light (such as red) and one or more configured to detect IR light. These photo detectors (for detecting light of different wavelengths) can be combined into a single photodiode or maintained separate from one another. Further, the one or more light sources and one or more photodetectors could reside in the same module (upper or lower) in the case of a reflective system or the light source(s) could reside in one module while the optical detector(s) reside in the other in the case of a transmissive system.

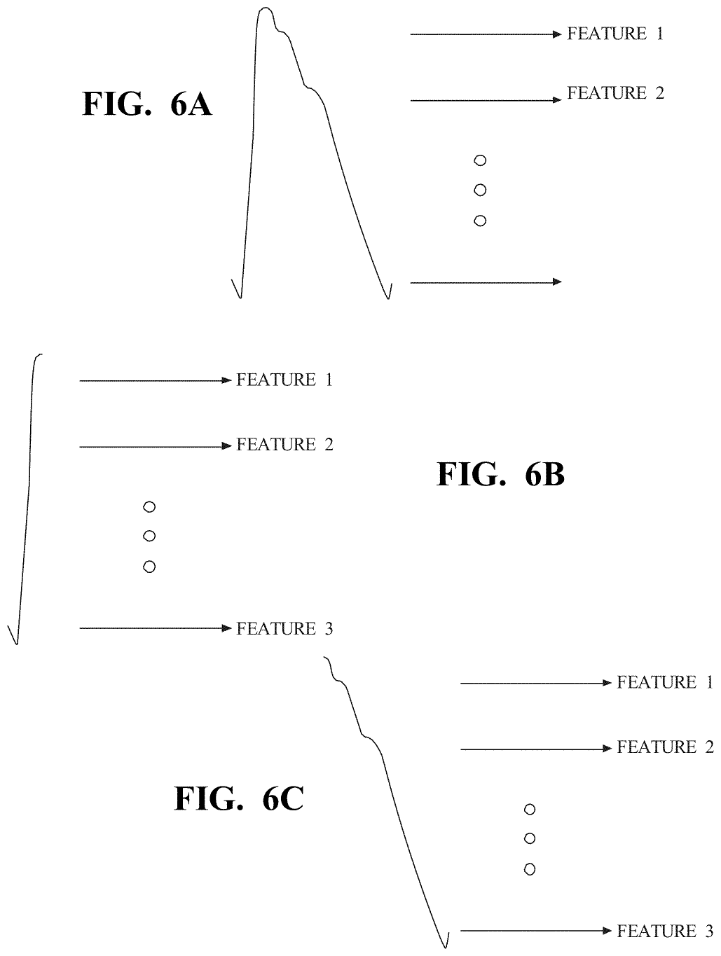

Upon collection of a blood flow pulse signal, a number of parameters can be extracted from both single pulses and a waveform comprising multiple pulses. FIG. 6A depicts a single pulse from which a number of features or parameters can be extracted. Features or parameters extracted from a single pulse can include, but are not limited to, shape of the pulse, a maximum amplitude, a minimum amplitude, a maximum derivative, a time difference between main and secondary peaks, and integral through the entire extraction time (i.e., the area under the pulse). FIGS. 6B and 6C illustrate that even portions of a single pulse can be analyzed for feature extraction. Extracting features at this level of detail has a number of advantages, including the ability to capture a great number of pulse features and store each of those features digitally without having to retain the analog waveform. The result is a savings in storage requirements and ease of data transmission.

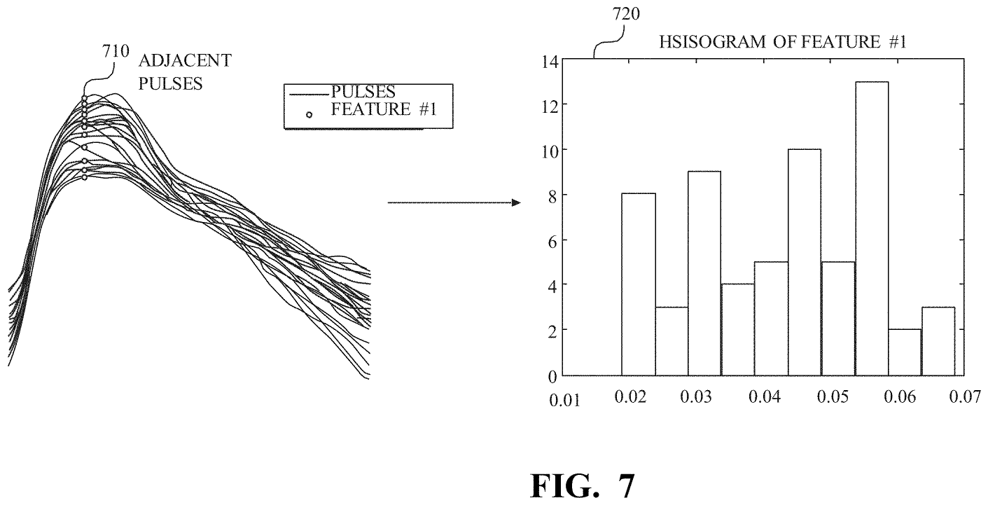

Feature extraction can also be performed on a number of pulses or a “pulse train.” FIG. 7 depicts a series of pulses overlaid with one another to show the variation among the group with respect to an identified feature according to implementations of this disclosure. In this manner, the total variation among a series of pulses with respect to a single feature can be determined. The average of a group of pulses with respect to a single feature and the standard deviation of the group with respect to the feature can also be determined. Of course, these are just examples of the types of information that can be collected from a comparison of a single feature over a group of pulses. Moreover, while FIG. 7 depicts the extraction of a single feature from the group of pulses, it should be appreciated that any number of features can be extracted from the group in a manner similar to that described above with respect to a single pulse. FIG. 7 further depicts how information collected about a single feature over a group of pulses can be digitized or presented in a histogram 720.

All of the features or parameters described above, collected using a PPG system comprising one or more light sources and/or one or more optical detectors, can be supplemented with additional sensors such as ECG electrodes/sensors, bio impedance sensors, galvanic skin response sensors, tonometry/contact sensors, accelerometers, pressure sensors, acoustic sensors, and electromagnetic sensors. For example, one or more tonometry/contact sensors can be used to extract tonometry information by measuring the contact vessel pressure. In another example, one or more acoustic sensors comprising a speaker-microphone combination (such as a micro-electro-mechanical system (“MEMS”) acoustic sensor) can be used to extract reflected sound pulses from moving vessel walls. Similarly, one or more electromagnetic sensor MEMS can be used to extract voltage induced by coils or magnet pieces pressed to moving vessel walls. In a further implementation, as described above, external or outward facing sensors can be configured to activate when touched by the off-hand (i.e., the hand on which apparatus 100 is not being worn) to collect additional information to help supplement or calibrate the information collected by the inward facing sensors of the upper or lower modules. For example, where internal facing PPG components (i.e., one or more light sources and one or more photo detectors) are used to detect reflected or transmitted light representative of blood flow pulses and some extrapolation of the data is made to determine, for example, heart rate, the user can place a fingertip of his or her off-hand on an outward facing ECG electrode (such as that shown in FIG. 1) to collect a more precise heart rate measurement. The more precise, though of more finite duration, heart rate measurement can be used to aid in the interpretation of the continuous heart rate measurements collected by the inward facing PPG sensors. The outward facing sensor can also comprise other sensors previously described herein, such as one or more contact/tonometry sensors, one or more bio impedance sensors, and one or more galvanic skin response sensors for analyzing electric pulse response. All of the information collected by an outward facing sensor from, for example, the fingertip of the user's off-hand, can be used to refine the analysis of the continuous measurements taken by any one or more of the inward facing sensors.

In addition to the inward and outward facing sensors, apparatus 100 may further comprise additional internal components such as one or more accelerometers and/or gyroscopic components for determining whether and to what extent the user is in motion (i.e., whether the user is walking, jogging, running, swimming, sitting, or sleeping). Information collected by the accelerometer(s) and/or gyroscopic components can also be used to calculate the number of steps a user has taken over a period of time. This activity information can also be used in conjunction with physiological information collected by other sensors (such as heart rate, respiration rate, blood pressure, etc.) to determine a user's caloric expenditure and other relevant information.

To determine a user's blood pressure, the PPG information described above may be combined with other sensors and techniques described herein. In one implementation, determining a user's blood pressure can comprise collecting a heart rate signal using a PPG system (i.e., one or more light sources and photo detectors) and performing feature extraction (described above) on single pulses and a series of pulses. The features extracted from single pulses and series of pulses can include statistical averages of various features across a series, information regarding the morphological shape of each pulse, the average and standard deviation of morphology of a series of pulses, temporal features such as the timing of various features within single pulses, the duration of a single pulse, as well as the average and standard deviation of the timing of a feature or duration of pulses within a series of pulses, and the timing of morphological features across a series of pulses (i.e., the frequency with which a particular pulse shape occurs in a series).

As described above, this feature extraction can not only be performed on a series of pulses and single pulses, but also on portions of a single pulse. In this manner, information pertaining to both systolic and diastolic blood pressure can be ascertained as one or more portions of an individual pulse correspond to the heart's diastole (relaxation) phase and one or more other portions of an individual pulse correspond to the heart's systole (contraction) phase. In some implementations, up to 200 features can be extracted from a partial pulse, a single pulse, and/or a series of pulses. In alternative implementations, fewer or more features may be extracted.

In addition to features extracted from PPG or ECG information, information and features can also be collected by contract/tonometry sensors, pressure sensors, bio impedance sensors galvanic skin response sensors, accelerometers, acoustic sensors, and electromagnetic sensors. For example, pressure sensors or bio impedance sensors can be used to identify blood flow pulses of user and, similar to PPG or ECG data, features can be extracted from the collected data.

The extracted features can then be cross-referenced or compared to entries in a library containing data corresponding to a population of subjects. For each subject, the library may contain information associated with each extracted feature. The library can also contain a direct measured or verified blood pressure for each subject. In further implementations, the library may contain more than one directly measured or verified blood pressure measurement for each subject, each corresponding to a subject in a different condition, such as one corresponding to the subject at rest, one corresponding to the subject engaged in light activity, and one corresponding to the subject engaged in strenuous activity. Thus, the extracted features of the user, as well as activity information pertaining to the user, can be compared to entries in the library to find one or more subjects with which the user's extracted features most closely match and the user's blood pressure can then be estimated based on the verified blood pressure of those subjects.

As one example, when features are extracted from a series of pulses, a standard deviation or range of variation across the series can be ascertained. Generally speaking, a large variation across a series of pulses can be associated with flexible, healthy veins. As a result, individuals exhibiting large pulse-to-pulse variations across a series of pulses typically have relatively low blood pressure. Conversely, little to no variation in features across a series of pulses is typically associated with relatively high blood pressure.

The library described above can be generated by extracting the same features from partial pulses, individual pulses, and series of pulses across hundreds or thousands of subjects. The subjects' verified blood pressure can also be measured such that it can be associated with each feature extracted from the subject's pulse information. The subject entries in the library can also be sorted based on information helpful for estimating blood pressure. For example, subjects in the library can be identified as male or female, belonging to a particular age group, or associated with one or more past health conditions. Individual subjects can be associated with information indicative of the subject's sex, age, weight, race, and any other medically meaningful distinction. Moreover, entries can be associated with information collected by other sensors at the time the verified blood pressure measurement was taken, including information collected by contract/tonometry sensors, pressure sensors, bio impedance sensors galvanic skin response sensors, accelerometers, acoustic sensors, and electromagnetic sensors. As just one example, if a user is determined to be engaged in physical activity (through a combination of accelerometer and heart rate data, as an example), his or her extracted features may only be compared to data in the library corresponding to subjects engaged in similar physical activity. Information pertaining to subjects contained in the library may also be correlated to each subject's resting heart rate, BMI, or some other medically significant indicia. For example, if a user is a young female with a low resting heart rate who is currently engaged in moderate activity, her extracted features should be compared to subjects in the library identified as young females with low resting heart rate whose blood pressure was verified during moderate activity rather than comparing the user's extracted features to an elderly male subject with a relatively high resting heart rate and whose blood pressure was verified during strenuous activity.

When the user's extracted features are compared to features recorded in the library, apparatus 100 can also weigh the entries of subjects most closely corresponding to the user more heavily than entries of subjects associated with indicia different from that of the user. For example, if the user is a male, features extracted from male subjects may be weighed more heavily than female subjects because a particular pulse variation in men of a particular age may correspond to relatively high blood pressure whereas the same pulse variation in women of that particular age may correspond to lower blood pressure.

According to the techniques described herein, accurate blood pressure estimates for a user can be made without requiring direct blood pressure measurement of the user. However, in some implementations, the user's blood pressure estimates can be further calibrated by direct measurement of the user's blood pressure by another device and that verified blood pressure can be input into apparatus 100 to aid in future estimations of the user's blood pressure.

Calibration can also be accomplished with an outward facing ECG sensor.

While an inward facing PPG sensor can continuously or periodically collect heart rate data of a user, occasionally the user may be prompted to place a fingertip of his or her off-hand on an outward facing ECG sensor (e.g., electrodes). The inward facing sensor arrays of apparatus 100 may contain additional electrodes thereby completing an electrical circuit through the user's body and allowing a more precise pulse waveform to be collected. Feature extraction can be performed on these pulses, series of pulses, and partial pulses in the same manner as described above with respect to PPG information and used to cross-reference the library.

In still a further implementation, where apparatus 100 determines, based on its continuous or periodic monitoring of the user's blood pressure using PPG or pressure sensors, that a user's blood pressure is unusually or dangerously high or low, apparatus 100 may prompt the user to place a fingertip of his or her off-hand on an outward facing ECG electrode in order to verify the unusual or unsafe condition. If necessary, apparatus 100 can then alert the user to call for help or seek medical assistance.

As described above, the upper and/or lower modules 110, 150 can be configured to continuously collect data from a user using its inward facing sensor arrays. However, certain techniques can be employed to reduce power consumption and conserve battery life of apparatus 100. For instance, in some implementations, only one of the upper or lower modules 110, 150 may continuously collect information. In alternative implementations, neither module may be continuously active, but may wait to collect information when conditions are such that accurate readings are most likely. For example, when one or more accelerometers or gyroscopic components of apparatus 100 indicate that a user is still, at rest, or sleeping, one or more sensors of upper module 110 and/or lower module 150 may collect information from the user while artifacts resulting from physical movement are absent.

While techniques for estimating a user's blood pressure using pulse signal, pressure, impedance, and other collected and input information has been described above, it should be appreciated that similar techniques can be employed to estimate a user's oxygen levels (SvO₂), hydration, respiration rate, and heart rate variability. For example, PPG, ECG, bio impedance, and acoustic measurements taken from the user can be cross-referenced with the aforementioned library and compared to subjects most closely matching the user (e.g., sex, age, height, weight, race, resting heart rate, BMI, current activity level, and any other medically meaningful distinction. Measured or verified hydration levels of one or more subjects can then be used to estimate the hydration level of the user. A similar process can be employed to estimate the user's oxygen levels (SvO₂), respiration rate, and heart rate variability.

FIG. 8 depicts an illustrative processor-based computing system (i.e., a system 800) representative of the type of computing system that may be present in or used in conjunction with any aspect of apparatus 100 comprising electronic circuitry according to implementations of this disclosure. Each of upper or lower modules 110, 150 may comprise any one or more components of system 800. In some implementations, one module may contain one of the components of system 800 and the other module, rather than comprising a similar component, may be in wired or wireless communication with the component residing in the other first module. Alternatively, each module may comprise a similar component as compared to the other module such that it is not necessary to communication with the first module to enjoy the functionality of the component. For example, upper module 110 may comprise storage, a power source, and/or a charging port, while lower module 150 may have access to the upper module's storage and/or draw power from the power source of the upper module through a wired or wireless connection. Alternatively, each module may have its own storage and/or power source. For the sake of simplicity, the system 800 will be described herein as if it encompasses the components of upper and lower modules 110, 150 collectively, while the reader appreciates that one or more components described herein may reside only in one module or may be found in both modules.

The system 800 may be used in conjunction with any one or more of transmitting signals to and from the one or more accelerometers, sensing or detecting signals received by one or more sensors of apparatus 100, processing received signals from one or more components or sensors of apparatus 100 or a secondary device, and storing, transmitting, or displaying information. The system 800 is illustrative only and does not exclude the possibility of another processor- or controller-based system being used in or with any of the aforementioned aspects of apparatus 100.

In one aspect, system 800 may include one or more hardware and/or software components configured to execute software programs, such as software for storing, processing, and analyzing data. For example, system 800 may include one or more hardware components such as, for example, processor 805, a random access memory module (RAM) 810, a read-only memory module (ROM) 820, a storage system 830, a database 840, one or more input/output (I/O) modules 850, an interface module 860, and one or more sensor modules 870. Alternatively and/or additionally, system 800 may include one or more software components such as, for example, a computer-readable medium including computer-executable instructions for performing methods consistent with certain disclosed implementations. It is contemplated that one or more of the hardware components listed above may be implemented using software. For example, the storage system 830 may include a software partition associated with one or more other hardware components of system 800. System 800 may include additional, fewer, and/or different components than those listed above. It is understood that the components listed above are illustrative only and not intended to be limiting or exclude suitable alternatives or additional components.

Processor 805 may include one or more processors, each configured to execute instructions and process data to perform one or more functions associated with system 800. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 8, processor 805 may be communicatively coupled to RAM 810, ROM 820, the storage system 830, database 840, I/O module 850, interface module 860, and one more of the sensor modules 870. Processor 805 may be configured to execute sequences of computer program instructions to perform various processes, which will be described in detail below. The computer program instructions may be loaded into RAM for execution by processor 805.

RAM 810 and ROM 820 may each include one or more devices for storing information associated with an operation of system 800 and/or processor 805. For example, ROM 820 may include a memory device configured to access and store information associated with system 800, including information for identifying, initializing, and monitoring the operation of one or more components and subsystems of system 800. RAM 810 may include a memory device for storing data associated with one or more operations of processor 805. For example, ROM 820 may load instructions into RAM 810 for execution by processor 805.

The storage system 830 may include any type of storage device configured to store information that processor 805 may need to perform processes consistent with the disclosed implementations.

Database 840 may include one or more software and/or hardware components that cooperate to store, organize, sort, filter, and/or arrange data used by system 800 and/or processor 805. For example, database 840 may include user profile information, historical activity and user-specific information, physiological parameter information, predetermined menu/display options, and other user preferences. Alternatively, database 840 may store additional and/or different information.

I/O module 850 may include one or more components configured to communicate information with a user associated with system 800. For example, I/O module 850 may comprise one or more buttons, switches, or touchscreens to allow a user to input parameters associated with system 800. I/O module 850 may also include a display including a graphical user interface (GUI) and/or one or more light sources for outputting information to the user. I/O module 850 may also include one or more communication channels for connecting system 800 to one or more secondary or peripheral devices such as, for example, a desktop computer, a laptop, a tablet, a smart phone, a flash drive, or a printer, to allow a user to input data to or output data from system 800.

The interface module 860 may include one or more components configured to transmit and receive data via a communication network, such as the Internet, a local area network, a workstation peer-to-peer network, a direct link network, a wireless network, or any other suitable communication channel. For example, the interface module 860 may include one or more modulators, demodulators, multiplexers, demultiplexers, network communication devices, wireless devices, antennas, modems, and any other type of device configured to enable data communication via a communication network.

System 800 may further comprise one or more sensor modules 870. In one implementation, sensor modules 870 may comprise one or more of an accelerometer module, an optical sensor module, and/or an ambient light sensor module. Of course, these sensors are only illustrative of a few possibilities and sensor modules 870 may comprise alternative or additional sensor modules suitable for use in apparatus 100. It should be noted that although one or more sensor modules are described collectively as sensor modules 870, any one or more sensors or sensor modules within apparatus 100 may operate independently of any one or more other sensors or sensor modules. Moreover, in addition to collecting, transmitting, and receiving signals or information to and from sensor modules 870 at processor 805, any one or more sensors of sensor module 870 may be configured to collect, transmit, or receive signals or information to and from other components or modules of system 800, including but not limited to database 840, I/O module 850, or the interface module 860.

As mentioned above, disclosed herein are also implementations of portable devices for obtaining ECG measurements of the heart. The ECG measurements can be used to monitor and/or detect heart abnormalities, such as ischemia.

In a conventional 12-lead ECG, ten electrodes are placed on the patient's limbs and on the surface of the chest. The overall magnitude of the heart's electrical potential is then measured from twelve different angles, which are referred to as “leads,” and is recorded over a period of time (usually ten seconds). In this way, the overall magnitude and direction of the heart's electrical depolarization is captured at each moment throughout the cardiac cycle.

The use of the chest electrodes is important to increase accuracy of diagnostics of cardiac disorders, such as ischemia.

In a portable device according to this disclosure, ECG sensors included in the portable device are augmented with navigation capabilities that can aid a user in making (e.g., taking) ECG measurements at the proper places of the body (e.g., the chest). Navigation, as used herein, can be defined as a way to identify a current location of the portable device on the body of the user. As such, the term navigation is used to describe the recognition of a current location of our portable device on the body of the user, for example based on a previous location.

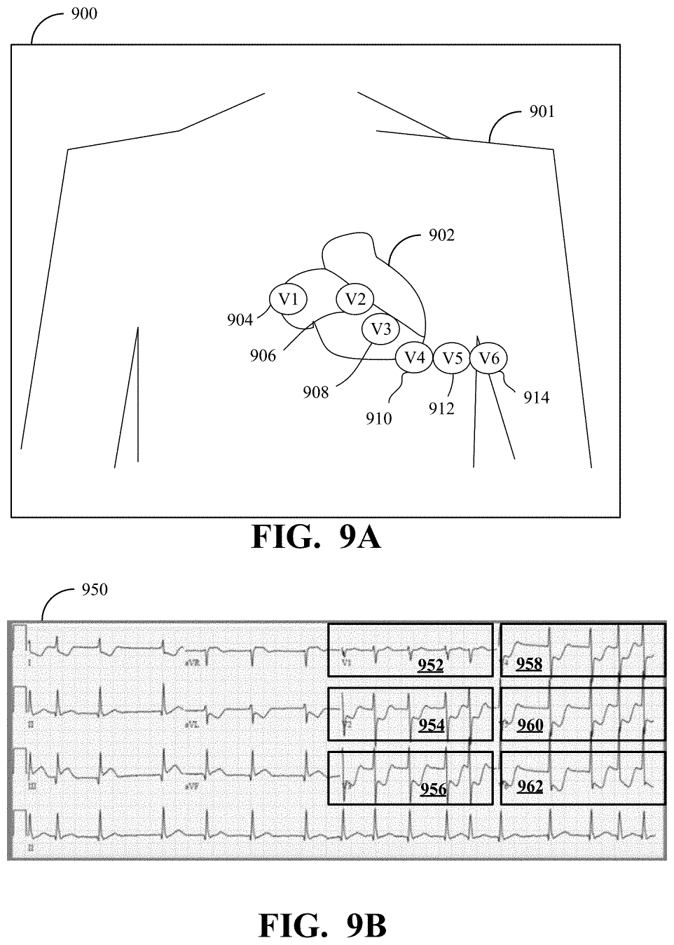

FIG. 9A illustrates placement 900 of precordial electrodes. In a traditional 12-lead ECG, six unipolar electrodes are placed on the chest. These electrodes are referred to as the precordial leads or electrodes. The placement 900 illustrates a torso 901 (e.g., chest) of a person whose ECG is to be measured. Six electrodes 904-914, typically referred to as the V1-V6 electrodes, are placed on the chest about the heart 902, as shown in FIG. 9A.

The electrode 904 is placed in the fourth intercostal space (between ribs 4 and 5) to the right of the sternum; the electrode 906 is placed in the fourth intercostal space (between ribs 4 and 5) to the left of the sternum; the electrode 908 is placed diagonally between electrodes 906 and 910 (i.e., between V2 and V4); the electrode 910 is placed in the fifth intercostal space (between ribs 5 and 6) in the midclavicular line; the electrode 912 is placed at the same level as the electrode 910, in the left anterior axillary line; and the electrode 914 is placed at the same level as the electrodes 910 and 912 in the midaxillary line.

The electrodes 904 and 906 are referred to as the septal leads; the electrodes 908 and 910 are referred to as the anterior leads; and the electrodes 912 and 914 are part of the anterolateral (or lateral) leads. The septal leads primarily observe the ventricular septum and may display ECG changes originating from the right ventricle of the heart; the anterior leads observe the anterior wall of the left ventricle of the heart; and the anterolateral leads observe the lateral wall of the left ventricle of the heart.

FIG. 9B illustrates regions of a traditional ECG 950 associated with each of the precordial electrodes. The horizontal direction of the traditional ECG 950 illustrates time. Regions 952-962 correspond, respectively, to ECG measurements of at the electrodes 904-914.

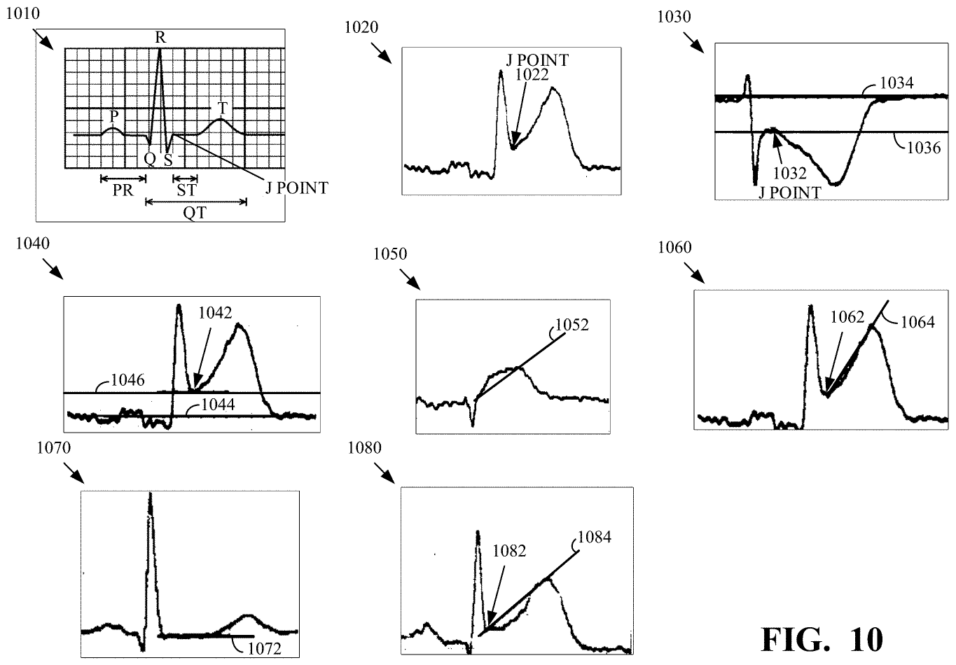

FIG. 10 illustrates examples of ECGs of normal and ischemic myocardium. A normal ECG 1010 illustrates the components of an ECG and the different waveforms. A P wave represents atrial depolarization. The P wave is followed by the QRS complex, which represents ventricular depolarization. The QRS complex is followed by the ST segment, which is followed by the T wave. The ST segment and T wave represent repolarization of ventricular tissue.

As is known, the overall direction of depolarization and repolarization produces positive or negative deflection on each lead's trace. The point at which ventricular depolarization ends and ventricular repolarization begins is referred to as the J point. Thus, the J point is the junction between ventricular depolarization and ventricular repolarization.

When the myocardium (i.e., the muscular tissue of the heart) is ischemic, changes can be observed on the ECG graph or by analyzing the ECG signals. Many of these changes can be isolated to the J point itself, the ST segment, and/or the T wave.

An ECG 1020 illustrates a first condition of an ischemic myocardium. In the ECG 1020, there's an acute angle at a J point 1022 that takes place at the end of the QRS complex and before the ST segment.

The J point may be evaluated against the isoelectric line (i.e., a horizontal line) to determine whether the ECG graph includes an ST segment elevation, ST segment depression, or a J point elevation. The isoelectric line represents a straight line on the ECG graph where there is no positive or negative charges of electricity to create deflections.

In an ECG 1030, an isoelectric line 1034 is the isoelectric line and a second line 1036 is drawn at the level of a J point 1032. As can be seen, the J point 1032 is below the isoelectric line 1034. The situation where the J point 1032 drops below the isoelectric line 1034 is referred to as ST segment depression (or more preferably J point depression). This situation is indicative of myocardial ischemia.

In an ECG 1040, an isoelectric line 1044 is the isoelectric line and a second line 1046 is drawn at the level of a J point 1042. In the ECG 1040, the J point 1042 is elevated compared to the isoelectric line 1044. This situation is referred to as ST-segment elevation (or more preferably J-point elevation). This situation is also indicative of myocardial ischemia.

In an ECG 1050, the shape of the ST segment is considered. In some ECGs, such as the ECG 1050, it may be difficult to identify the J point. A line 1052 is drawn from the end of the QRS complex to the tallest point of the T wave. As can be seen, at least part of the ST segment or T wave lies above the line 1052, which is most consistent with ischemia. This situation is referred to as convex ST segment. Convex ST segments are indicative of ischemic abnormalities.

In an ECG 1060, the ST segment is flat and a J point 1062 is fairly clearly identifiable. A line 1064 is drawn from the J point 1062 to (e.g., through) the tip of the T wave. As can be seen, the ST segment or an early phase of the T wave lie on the line 1064. Flat ST segments or T waves are also indicative of ischemia. An ECG 1070 also illustrates a flat ST segment as illustrated by a line 1072 that is drawn from the end of the QRS complex through the ST segment.

In an ECG 1080, a line 1084 is drawn from a J point 1082 toward the tip of the T wave. As can be seen in the ECG 1080, the ST segment falls below the line 1084. That is, the ST segment is said to be concave. While concave ST segments do not necessarily identify ischemia, that is not always conclusive. Thus, it is possible that a concave ST segment may be due to an ischemic change.

While FIG. 10 described some ECG characteristics associated with ischemia, the described characteristics are not exhaustive. For example, ischemia can be identified based on characteristics associated with the T wave. For example, a peaked (e.g., tented) T wave is associated with ischemia. A normal T wave typically has a gradual upslope and a rapid return to a baseline. However, a T wave that may resemble a teepee or is peaked or tented is characteristic of ischemia. As another example, a T wave with a broad base can also be characteristic of ischemia.

By measuring ECGs at different locations of the chest (e.g., at the V1-V6 locations) and analyzing features of the ECGs, ischemic conditions can be identified in different parts of the myocardium. Extracting features from ECG signals is familiar to a person skilled in art and it thus omitted here.

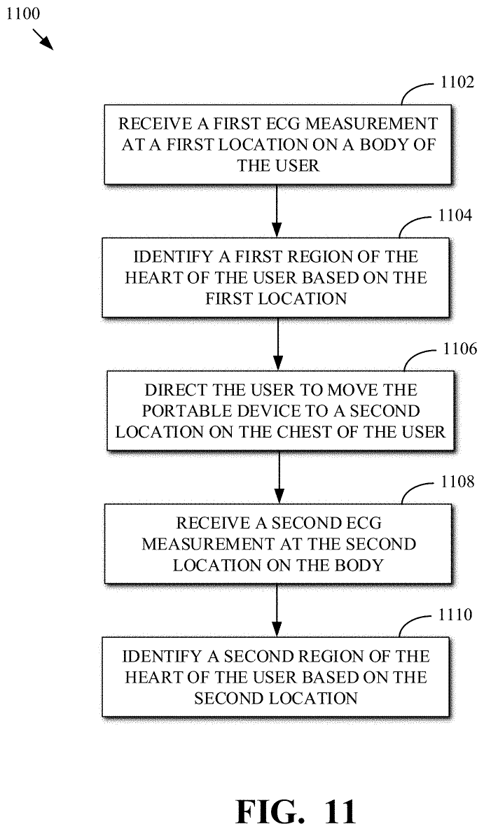

FIG. 11 is a flowchart of a technique 1100 for measuring an ECG of a user according to an implementation of this disclosure. The technique 1100 can be implemented as executable instructions that can be stored in a memory, such as one or more of the storage system 830 or the ROM 820 of FIG. 8. The instructions can be executed by a processor, such as the processor 805 of FIG. 8, to perform the steps of the technique 1100. The technique 1100 can be implemented using specialized hardware or firmware.

The technique 1100 can be implemented by a portable device. The portable device can be a module, such as the upper module 110 or the lower module 115 of FIG. 1. The portable device can include a first ECG sensor that the user can place on different locations of the user's chest, such as one or more of the locations V1-V6 identified with respect to FIG. 9A. The portable device can have many possible configurations, a few of which are described below.

In an example, the portable device can be a device that the user can hold between his/her fingers to place the first ECG sensor at one of the locations V1-V6 identified with respect to FIG. 9A. The portable device may or may not be attached to a strap. In a case that the portable device is attached to a strap and is wrist-worn, the user may unfasten the strap and hold the portable device between his/her fingers to place the first ECG sensor on the chest. Thus, the portable device can include a front side and a back side that is opposite the front side. The back side is the side that is intended to be placed on the body of the user. The back side includes the first ECG sensor.

While not shown in the figures, such as for example FIG. 2, the portable device can include at least one second ECG sensor. In an example, the at least one second ECG sensor can be included in one of the lateral sides of the portable device (referred to herein as “side sensors”) so that when the user is holding the portable device between the fingers, at least one of the fingers is in contact with the at least one second ECG sensor. The lateral sides of the portable device are, for example, those sides that are generally perpendicular to the side that includes the first ECG sensor.

In an example, the portable device includes one side sensor. In another example, the portable device includes two side sensors. In an example, the two side sensors can be placed on adjacent lateral sides of the portable device. In an example, the side sensors can be placed on opposing lateral sides of the portable device.

In an example, the portable device can include a second ECG sensor that is disposed in a side that is opposite (i.e., referred to herein as the “front sensor”) the side that includes the first ECG sensor. Thus, for example, while the user is holding the portable device between his/her fingers, such as the thumb and the middle finger, the user can place the index finger on the front sensor. In an example, the portable device can include the first ECG sensor, at least one side sensor, and a front sensor. In an example, the portable device can include the first ECG sensor, at least two side sensors, and a front sensor.

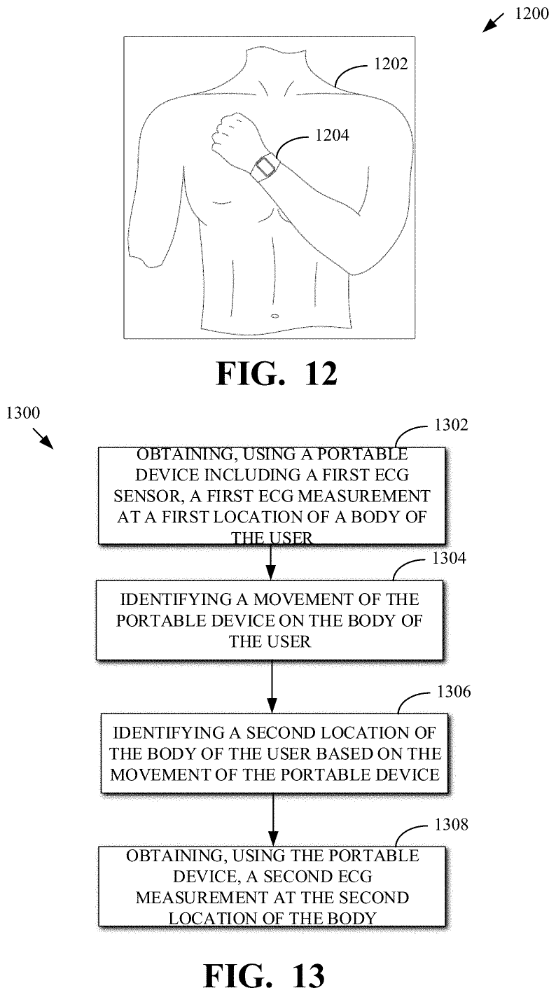

In another example, the portable device can be a wrist-worn device, which the user can place on the his/her chest while the portable device is worn by the user. The first ECG sensor can be included in a lower module, such as the lower module 150 of FIG. 1. The ECG sensor can be disposed on a side of the lower module that is opposite the side that includes the sensor array 155. That is, the first ECG sensor is included in the side that does not face (e.g., touch, etc.) the wrist of the user. In another example, the portable device may not include a lower module. Thus, the first ECG sensor can be included (e.g., disposed, etc.) in a buckle of the strap of the portable device. As such, to take an ECG measurement at one or more of the locations V1-V6, the user places his/her wrist proximal to the one or more locations V1-V6, as shown in FIG. 12. FIG. 12 illustrates an example 1200 of a user 1202 placing a wrist-worn portable device 1204 at the V1 location of FIG. 9A according to implementations of this disclosure.

In an example, the portable device can include at least one second ECG sensor. In an example, the portable device can include a first second ECG sensor in the sensor array 155 and a second second ECG sensor in an upper module, such as the upper module 110 of FIG. 1. Thus, when taking a measurement, the user can place the first ECG sensor on his/her chest, the first second ECG sensor can touch the wrist of the user, and the user can place an a finger on the second second ECG sensor.

In an example, the portable device can include at least one second ECG sensor in the tail of a strap, such as the strap or band 105 of FIG. 3, of the portable device (referred to herein as a strap sensor). For example, the strap sensor may be disposed in a tail of the strap. The tail can be long enough so that the at least one second ECG sensor can be accessible to the user while the wrist of the user is placed on the chest. For example, the user may wear the portable device on his right/left wrist, and as the user places his/her the right/left wrist, the user can hold the at least one second ECG sensor between the thumb and index fingers of the other left/right hand. Thus, to reiterate, the portable device can be configured to be worn on a wrist of a first arm of the user, the tail of the strap can include a second ECG sensor, and the second ECG sensor can be configured so that the user can hold the second ECG sensor using fingers of the second arm of the user.

In another example, the at least one second ECG sensor can be disposed on the strap of the portable device. In an example, the at least one second ECG sensor can be disposed on one side of the lower module. In another example, the at least one second ECG sensor can be disposed on the strap on both sides of lower module. As such, the at least one second ECG sensor can include at least two strap sensors, which the user can touch while the lower module is placed on the chest or other parts of the user's body.

In an example, the current placement (e.g., a current location) of the portable device on the body of the user can be determined based on a displacement from a previous location of the portable device on the body. For example, the portable device may be previously placed at the V1 location. That the portable device is currently placed at the location V3 can be determined by determining (e.g., calculating, measuring, detecting, observing, etc.) the displacement from the V1 location to the current location.

The displacement can be determined in several ways. In an example, the portable device can include one or more sensors (referred to herein as navigation sensors) that can be used to measure the displacement of the portable device on the body (e.g., chest) of the user. To reiterate, data from the navigation sensor can be used to measure the direction and/or length of displacement when the user moves the navigation sensor (e.g., the portable device) along the user's body while or before pressing the portable device to the skin. In another example, the displacement can be determined using a device that is external to the portable device.

As mentioned, the portable device can include navigation sensors. Any number of one or more navigation sensor can be used. Examples of navigation sensors include, but are not limited to, LEDs and photodiodes, an array of photosensors (e.g., minicameras, an optoelectronic sensor) with optional additional light(s), a mechanical sensor with a rolling (e.g., track) ball, or an ultrasound sensor.

For example, with respect to the mechanical sensor with a rolling ball, the rolls (e.g., movements, etc.) of the track ball can be converted to an angle and a distance of displacement on the body. For example, with respect to the optoelectronic sensor, successive images of the surface of the chest on which the portable device is moved are taken by the optoelectronic sensor to determine the angle and distance of displacement. Differences between the successive images are used to determine the displacement. For example, in the case of LEDs and photodiodes, several (e.g., 200, more, or fewer) measurements of the white level at one point can be taken and based on changes in the level of luminosity, the displacement can be determined. For example, with respect to the an ultrasound sensor, differences between the successive sound reflections are can be to determine the displacement. That is, in the case of an ultrasound transceiver and receiver, several (e.g., 200, more, or fewer) measurements of the sound reflections at one point can be taken, and based on changes in the intensity of reflection (e.g., the echo), the displacement can be determined.

In another example, the navigation sensors can be or can include an accelerometer. Accelerometer data can be used to determine a displacement from a previous location to a current location of the portable device on the body of the user. In another example, a gyroscope can be additionally be used.

In another example, the displacement can be determined using a device that is external to the portable device. The external device can be a device that is in communication with the portable device. The external device can be, for example, a mobile phone or the like, of the user and that is in communication with the portable device. When the user is ready to take ECG measurements, the external device is placed by the user in front of the user so that the external device (e.g., sensors therein) can perceive the location of the portable device on the body of the user.

The portable device and the external device can communicate via wired or a wireless connection. A wired connection can be a Universal Serial Bus (USB) connection, a firewire connection, or the like. A wireless connection can be via a network using Bluetooth communications, infrared communications, near-field communications (NFCs), a cellular data network, or an Internet Protocol (IP) network. In an example, the external device can communicate the location of the portable device on the body of the user to the portable device. In another example, the portable device can receive raw sensor data from the external device and the portable device can process the raw sensor data to determine a location of the portable device on the body of the user and/or a displacement of the portable device.

In an example, the external device can include a camera, which can be used to take images of the placement of the portable device on the body of the user. Image processing can be used to determine the displacement of the portable device between a first image and a second image. Alternatively, or equivalently, image processing can be used to determine a current location of the portable device on the body of the user. For example, an image can be compared to a stored image that includes a known location (e.g., V1-V6 or some other ECG sensor location). In an example, the stored image can be taken during a calibration process of the portable device.

In another example, the external device can be or can include one or more ranging and distancing sensors. For example, the external device can include a LiDAR sensor or a RADAR sensor that can be used to determine the location of the portable device on the body of the user. For example, short impulses of light or radio energy can be transmitted, reflected off the body, and then returned as an echo. The sensor location and its displacement can be determined by analyzing the echo with any technique known in the art for analyzing echo.

Returning to FIG. 11, at 1102, the technique 1100 receives a first ECG measurement at a first location on a body of the user. The first ECG sensor can be placed by the user at the first location of the body. The first location can be any location of the body where an ECG lead is typically measured. For example, the first location can be, or can be proximal, one of the V1-V6 locations of FIG. 9A.

In an example, the user can be prompted (for example, by the portable device, by the external device if available, or in some other way) to place the portable device to different points on the body according to a scheme, such that the scheme of FIG. 9A. A scheme, as used herein, refers to a pattern or a set of locations on the body of the first ECG sensor. The user can press the first ECG sensor to the skin and hold for a period of time (e.g., 5 seconds, 10 seconds, shorter, or longer period of time). The portable device collects ECG measurements at the current location for the period of time at a predefined frequency. The measurements (e.g., features extracted from each of the ECG signals withing the period of time) can be processed (e.g., to exclude noisy or outlier signals) and/or aggregated (e.g., averaged, etc.). The technique 1100 can determine the location of the portable device on the body. Equivalently, the technique 1100 can determine the ECG lead associated with the placement of the portable device on the body.

In an example, the user can be prompted to place the portable device with the first ECG sensor to some specific point of the body. For example, the user can be prompted to place the ECG sensor between V1 and V2 of FIG. 9A. Several ways may be available for prompting the user for an initial placement of the portable device during a measurement episode. A measurement episode is defined as a set of measurements at different locations that are intended to constitute a measurement.

For example, in one sitting, in a measurement episode, the user may take V1-V6 lead measurements. The user may be instructed via help documentation of the portable device that the initial placement should be between V1 and V2. The user may be visually shown (such as on a display of the portable device and/or a display of an external device that may be in communication with the portable device) where to place the portable device on the body. For example, the user may be told, with a verbal message to “place the device between V1 and V2,” or the like.

In an example, the user can be prompted to place the ECG sensor at a reference point of the body. The reference point can be used as the initial reference point for calculating subsequent displacements for identifying the locations of the portable device on the body. The reference point can be set during a calibration process, which is described below.

In an example, the initial (e.g., first) location (e.g., reference point) can be identified based on a remembered wave shape of an ECG wave or based on local electrical or optical properties of the skin at the initial location. In another example, a camera of the portable device, if available, can be used to take an image of a local skin area. Using visible skin structures (e.g., scars, moles, nevus, etc.) in the image, the device can recognize the placement. In yet another example, the user can be prompted to place the portable device onto a unique and easily identifiable location on the body, such as above the navel.

In an example, where the user places the portable device can be assumed to match the prompted location. This initial location can be used, via displacements, to identify subsequent locations.

In the case that, as described above, an external device that includes, for example, a camera is available, the first location of the portable device can be recognized by comparing stored images of the expected V1-V6 locations (i.e., stored images of the user with the portable device placed at each of the V1-V6 locations or other body locations) to a current image that is taken by the camera of the current location of portable device on the body of the user.

At 1104, the technique 1100 identifies a first region of the heart of the user based on the first location. That is, based on the first location (e.g. which of V1-V6), the technique 1100 can identify the region of the heart to which the measurement correlates, as described with respect to FIGS. 9A-9B.

At 1106, the technique 1100 directs the user to move the portable device to a second location on the body of the user. After a measurement is taken at a current location, the user moves the portable device according to the scheme (e.g., FIG. 9A).

In a first example, which for ease of reference is referred to as passive navigation, the user can place the portable device onto any location of the body and start moving the portable device from point (e.g., location) to point. The initial location having been determined (e.g., recognized, identified, etc.), information from the navigation sensors of the portable device can be used to compare the scheme of the displacements with the predefined points (such as the scheme of FIG. 9A). A current point (e.g., location) of measurement can then be recognized.

In an example, the user can be notified with a success signal (e.g., a sound, a haptic tap, a vibration, etc.) that the measurement at the current location is completed. In an example, the portable device can notify the user that the measurement was not successful with a failure signal. The measurement may not be successful because, for example, the ECG shape was not recognized in the signal that is received from the first ECG sensor. The ECG shape can be said to be recognized when the ECG shape matches stored normal or ischemic ECG shapes, as described with respect to FIG. 10. In an example, the portable device can notify the user that the point of the measurement was not recognized.

The user can then move the portable device along the body to the different points of the scheme (e.g., V1, V2, V3, V4, V5, V6), presses the first ECG sensor to the skin, and holds the portable device for the period of time. The user may move the portable device along the sequence V1, V2, V3, V4, V5, and V6 or some other random order. The portable device (e.g., a module therein) can compare the pattern of the movements, based on displacement information (e.g., displacement calculations), with the expected route (e.g., scheme) and sorts the points measurement in the order of the scheme (e.g., V1 through V6).

In a second example, which for ease of reference is referred to as active navigation, the user can be prompted to place the first ECG sensor at any point (e.g., location) of the body. The technique 1100 can compare the data from the navigational sensors with the locations of the scheme. If a location according to the scheme is recognized, then an ECG measurement is performed. If the technique 1100 recognizes an improper placement (e.g., a placement of the portable device that does not correspond to any of the locations of the scheme), then the user can be notified of the improper placement, such as via a voice, a visual, a haptic, or the like message. The user can then be asked to move the first ECG sensor (e.g., to move the portable device) to the proper location on the body. To illustrate, a voice command may state “move the device 2 cm to the right.” After the measurement, the user is instructed (e.g., prompted, etc.) with the next command to move the portable device to another location.

In an example, the technique 1100 can notify the user that the portable device is at a location where a measurement should be taken. For example, as the user moves the portable device from a current measurement location to a next measurement location, the technique 1100 can compare, using navigation sensor information, whether an intermediate location is one of the possible next measurement locations according to the scheme. For example, an audible signal can be emitted from the portable device as the user moves the device. The audible signal can change (e.g., in pitch, volume, intermittency, etc.) as the user gets closer to a measurement location at which an ECG measurement has not been taken. The user can recognize based on the audible signal to stop moving the portable device and to press and hold the portable device to the skin at the location so that an ECG measurement can be taken by the technique 1100. In another example, the user can be directed with verbal messages. For example, a message may direct the user to “stop here.”

As such, in an example, directing the user to move the portable device to the second location on the body of the user can include notifying the user that the portable device is at the second location. In an example, notifying the user that the portable device is at the second location can include comparing intermediate locations of the portable device to predefined locations until the second location is identified.

At 1108, the technique 1100 receives a second ECG measurement at the second location on the body. At 1110, the technique 1100 identifies a second region of the heart of the user based on the second location, as described above with respect to 1104.

In a typical 12-lead ECG setting, all electrodes are connected/affixed to the body at the same time and measurements of all of the 12 leads are taken simultaneously.

In implementations according to this disclosure, heart beats recorded within a short time and in the same conditions can be assumed to have the same ECG properties and can be considered to be measurements of the same (e.g., one) heartbeat. In another example, statistics on the heartbeats can be collected. For example, the average properties of the heartbeats at different (e.g., every) location of the body can be used to describe the measurements statistically (e.g., average, standard deviation, etc.). As such, that measurements at different locations are not taken simultaneously would not impact the results.

In an example, the respective locations of the scheme (i.e. the respective locations V1-V6) can be estimated based on a calibration process. For example, during an initial setup process or at any other calibration time, the user can be asked to provide information regarding the user's body type. For example, the user may be asked to identify whether the user is male or female; to identify whether the user has a lean and delicate body build, has a compact and muscular body build, or has soft round build of body and a high proportion of fat tissue; to identify body measurements (such as neck size, chest size, height, or the like); to identify fewer, more, other information, or a combination thereof. Such information can be used to estimate the respective locations of the scheme on the body as related to one reference point on the body. The locations can be estimates based on data collected from many people.

In another example, during a calibration process, the user may be asked to place the portable device at a first location of the body (e.g., the left or right arm pit or the V6 location) and then at a second location (e.g., the Xiphoid process, which is the most distal edge of the sternum). The respective locations can then be estimated based on the first location and the second location. At least one of the first location or the second location can be set as a reference point.

In an example, during a calibration process, the respective shapes of the ECG signals at each of the locations of the scheme can be stored. When the user subsequently measures an ECG at a current location, the shape of the ECG at the current location can be compared to the stored shapes of the ECG signals to identify the closest matching stored shape. The location of the scheme can be identified as the location of the matched stored shape. Herein, the shape of the ECG can refer to, or can be determined from, features extracted from the ECG signal. In an example, this shape-matched location identification can be combined with the above described navigation-based location identification to identify the location of the scheme. In an example, the location of the scheme may be identified as the location that is between the shape-matched location and the navigation-based location. In an example, the location of the scheme may be identified as the location that is closest to either the shape-matched location or the navigation-based location.

It is to be noted that it is not essential that the user uses a strict set of the points according to the scheme (e.g., FIG. 9A). The user can make the measurements in random points along a route (i.e., a user route). The portable device can store the scheme of the points (e.g., the user route). In a subsequent measurement episode, in the case of passive navigation, the user can make the measurements along approximately the same route, then the technique 1100 can compare the navigational sensor data with stored data to sort the measurements with the scheme of the last route. The specific locations of the measurements can be recognized and if the measurements were performed by the user in an order that is different from the initial (e.g., expected, previous, etc.) order, the measurements can be sorted according to the recognized (e.g. expected) locations. In the case of active navigation, the user can place the portable device into the first point (e.g., the reference point) or at the beginning of the last route, and the technique 1100 can guide the user as to how to move the portable device to the next measurement point. For example, the technique 1100 can cause the portable device to omit a message stating “stop here” when the user moves to location where a previous (e.g., stored, etc.) measurement was taken.

The scheme of measurements does not have to satisfy the scheme described with respect to FIG. 9A. The scheme of FIG. 9A is typically used in clinical settings. It can be sufficient to randomly measure ECGs at different points around the heart so long as a sufficient area around heart is covered by measurements so that abnormalities can be detected. Sufficient area in this context refers to a total number of measurements (e.g., 10, 15, 20, etc.) around the heart. Measurements, overtime, at the same locations around the heart can be used to detect changes in the ECG shapes at the different locations thereby identifying potential deteriorations in the heart muscles.

In an example, the sufficient area can be defined by points on an invisible grid with, for example, 5 cm between the points of the grid. The grid can be such that it covers half of the chest. The data about ECG shapes at the grid locations can be analyzed over time. In an example, the data can be transferred from the portal device to another system where the analysis can be performed. The technique 1100 can use active or passive navigation to navigate the user over the points to grid to take measurements.

In an example, the technique 1100 can store the points (e.g., locations) of the measurements of a measurement episode and corresponding ECG shapes (e.g., features of the ECG signals). The technique 1100 can store the locations and features in a memory of the portable device and/or can transfer the locations and features to another device that is communication with the portable device. In a subsequent measurement episode, the technique 1100 can guide the user to move the first ECG sensor into the same stored locations. New ECG shapes corresponding to new measurements at the same stored locations can be recorded. The new ECG shapes can be compared to the previous recorded shapes (e.g., features) to make conclusions regarding their similarity and/or alert the user to any detected abnormalities and changes. The comparison can be performed by the technique 1100. In another example, the comparison can be performed by the device to which the measurements are transferred. If there is sufficient similarity, then the data can be transferred to a diagnostic system. If no sufficient similarity is found for at least one location, the user can be prompted to repeat the measurement at the at least one location. In another example, If no sufficient similarity is found for at least one location, the user can be asked to repeat all measurements along the same route as the stored locations.

The ECGs of a measurement episode can be displayed as a graph that is similar to a standard ECG plot, as shown in FIG. 9B.

FIG. 13 is a flowchart of another technique 1300 for measuring an ECG of a user according to an implementation of this disclosure. The technique 1300 can be implemented as executable instructions that can be stored in a memory, such as one or more of the storage system 830 or the ROM 820 of FIG. 8. The instructions can be executed by a processor, such as the processor 805 of FIG. 8, to perform the steps of the technique 1300. The technique 1300 can be implemented using specialized hardware or firmware. The technique 1300 can be implemented by a portable device, which can be as described with respect to FIG. 11.

At 1302, the technique 1300 obtains, using the portable device that includes a first ECG sensor, a first ECG measurement at a first location of a body of the user.

At 1304, the technique 1300 identifies a movement of the portable device on the body of the user. In an example, identifying the movement of the portable device on the body of the user can include obtaining, using a navigation sensor of the portable device, a displacement of the portable device from the first location to the second location, as described above. In an example, the navigation sensor can be, or can include, an accelerometer. In an example, the navigation sensor can be, or can include, an optoelectronic sensor. In an example, identifying the movement of the portable device on the body of the user can include identifying previously stored locations and corresponding ECG measurements and identifying the second location of the body based on a comparison of the second ECG measurement to the corresponding ECG measurements, as described above.

At 1306, the technique 1300 identifies a second location of the body of the user based on the movement of the portable device. The first location and the second location can be locations according to a scheme for obtaining ECG measurements. As described with respect to FIG. 9A, the scheme can include a set of points on a chest of the user.

In another implementation, a system for measuring an electrocardiogram (ECG) of a user includes a portable device that includes a first ECG sensor and an external device that is communication with the portable device. The portable device can be as described above.

The portable device can include instructions stored in a memory to obtain a first ECG measurement at a first location of a body of the user, identify the first location based on sensor information received from the external device, prompt the user to move the portable device to a second location of the body of the user, and obtain a second ECG measurement at the second location of the body.

The external device can include instructions to obtain, using a camera of the external device, an image of the user holding the portable device to the body of the user; and compare the image to stored images to identify the first location.

Identifying, by the portable device, the first location based on the sensor information received from the external device can include receiving, from the external device, the first location.

While implementations have been illustrated and described, it will be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A portable device for measuring an electrocardiogram (ECG) of a user, comprising:
a first ECG sensor, wherein the first ECG sensor is configured to touch a body of the user;
a processor, the processor configured to:
receive a first ECG measurement at a first location on the body of the user, wherein the first ECG sensor is placed at the first location of the body;
identify a first region of the heart of the user based on the first location;
direct the user to move the portable device to a second location on the body of the user;
receive a second ECG measurement at the second location on the body; and
identify a second region of the heart of the user based on the second location.

2. The portable device of claim 1, further comprising:
a front side and a back side opposite to the front side, wherein the back side comprises the first ECG sensor.

3. The portable device of claim 1, further comprising:
a strap, wherein the strap is attachable to the portable device.

4. The portable device of claim 3, wherein the first ECG sensor is disposed in a buckle of the strap.

5. The portable device of claim 4,
wherein the portable device is configured to be worn on a wrist of a first arm of the user,
wherein a tail of the strap comprises a second ECG sensor, and
wherein the second ECG sensor is configured so that the user holds the second ECG sensor between fingers of the second arm of the user.

6. The portable device of claim 1, wherein to direct the user to move the portable device to the second location on the body of the user comprises to:
notify the user that the portable device is at the second location.

7. The portable device of claim 6, wherein to notify the user that the portable device is at the second location comprises to:
compare intermediate locations of the portable device to predefined locations until the second location is identified.

8. The portable device of claim 1, further comprising:
a navigation sensor, wherein the navigation sensor is used to identify a location of the portable device on the body of the user.

9. The portable device of claim 8, wherein the navigation sensor is an accelerometer.

10. The portable device of claim 8, wherein the navigation sensor is an external device that is in communication with the portable device, the external device including a camera, and wherein the location of the portable device on the body of the user is determined using images taken by the camera.

11. A method for measuring an electrocardiogram (ECG) of a user, comprising:
obtaining, using a portable device comprising a first ECG sensor, a first ECG measurement at a first location of a body of the user;
identifying a movement of the portable device on the body of the user;
identifying a second location of the body of the user based on the movement of the portable device; and
obtaining, using the portable device, a second ECG measurement at the second location of the body.

12. The method of claim 11, wherein identifying the movement of the portable device on the body of the user comprises:
obtaining, using a navigation sensor of the portable device, a displacement of the portable device from the first location to the second location.

13. The method of claim 12, wherein the navigation sensor is an accelerometer.

14. The method of claim 12, wherein the navigation sensor is an optoelectronic sensor.

15. The method of claim 11, wherein the first location and the second location are locations according to a scheme for obtaining ECG measurements.

16. The method of claim 15, wherein the scheme includes a set of points on a chest of the user.

17. The method of claim 11, wherein identifying the movement of the portable device on the body of the user comprises:
identifying previously stored locations and corresponding ECG measurements; and
identifying the second location of the body based on a comparison of the second ECG measurement to the corresponding ECG measurements.

18. A system for measuring an electrocardiogram (ECG) of a user, comprising:
a portable device, comprising a first ECG sensor; and
an external device that is communication with the portable device, the portable device is configured to:
obtain a first ECG measurement at a first location of a body of the user;
identify the first location based on sensor information received from the external device;
prompt the user to move the portable device to a second location of the body of the user; and
obtain a second ECG measurement at the second location of the body.

19. The system of claim 18, wherein the external device is configured to:
obtain, using a camera of the external device, an image of the user holding the portable device to the body of the user; and
compare the image to stored images to identify the first location.

20. The system of claim 19, wherein to identify the first location based on the sensor information received from the external device comprises to:
receive, from the external device, the first location.
